(19) United States
(12) Patent Application Publication
(10) Pub. No.: US 2022/0377223 A1
(43) Pub. Date: Nov. 24, 2022

(54) HIGH PERFORMANCE BRIGHT PUPIL EYE TRACKING

(71) Applicant: Seeing Machines Limited, Australian Capital Territory (AU)

(72) Inventors: John NOBLE, Australian Capital Territory (AU); Fergus MCCRACKEN, Australian Capital Territory (AU)

(21) Appl. No.: 17/769,726

(22) PCT Filed: Nov. 6, 2020
(86) PCT No.: PCT/AU2020/051214

§ 371 Date: Apr. 15, 2022

(30) Foreign Application Priority Data
Nov. 7, 2019 (AU) 2019904202
Mar. 17, 2020 (AU) 2020900811

(51) Int. Cl. H04N 5/235; G06T 7/246

(52) U.S. Cl. CPC H04N 5/2352; H04N 5/2351; G06T 7/246; G06V 20/597

(57) ABSTRACT

Described herein is a method (800) and system for controlling one or more illumination devices in an eye tracker system (100) such that a measured pupil/iris contrast exceeds a predefined minimum pupil/iris contrast. The method (100) includes: a. capturing images of a subject (102), including one or both of the subject's eyes, during predefined image capture periods; b. illuminating, from one or more illumination devices (108 and 110), one or both of the subject's eyes during the predefined image capture periods, wherein at least one illumination device (108 and 110) is located sufficiently close to a lens of the camera to generate bright pupil effects; and c. selectively varying the output power of at least one of the illumination devices (108 and 110) to generate a bright pupil reflection intensity such that a measured pupil/iris contrast in a captured image exceeds a predefined minimum pupil/iris contrast.

### FIELD OF THE INVENTION

The present application relates to illumination device control and in particular to an illumination system for an eye tracker.

Embodiments of the present invention are particularly adapted for illuminating a subject's face to extract facial features such as a pupil under bright pupil conditions. However, it will be appreciated that the invention is applicable in broader contexts and other applications.

### BACKGROUND

Many eye tracking systems rely on detection of pupil contours to accurately track eye gaze of a subject. To detect these contours, there must exist an identifiable contrast between an imaged pupil and a surrounding iris.

Where an illuminator is located far from an optical axis of an imaging camera, pupils appear dark and a large contrast exists between a pupil and the surrounding bright iris.

Conversely, when an illuminator is located close to the optical axis of an imaging camera (typically within about 3.25 degrees from the camera optical axis), the pupil can appear bright due to retroreflection by the eye. In traditional photography, this manifests as the well-known ‘red-eye’ effect due to the optical characteristics of the internal eye regions. In infrared imaging, where greyscale images are generated, this manifests as a ‘bright pupil’ effect in which the pupil appears white or almost white and typically brighter than the iris.

Eye tracking can be performed using a camera and illuminators under either bright or dark pupil conditions. Under dark pupil conditions, a pupil contrast is typically higher, making eye tracking more accurate. However, for typical dash-mounted driver monitoring systems, the illuminator or illuminators are required to be located at least about 2 cm from the camera in order to achieve dark pupil conditions when operating at wavelengths of 950 nm. This translates directly to a larger size camera and illuminator assembly. With the increase in vehicle electronics, the space available on a vehicle dash is increasingly becoming expensive and this trend is anticipated to continue with the advent of semi-autonomous vehicles.

As such, there is a commercial desire to minimize the spatial footprint of a driver monitoring system in a vehicle. For this reason, it may be advantageous to implement bright pupil eye tracking systems in future.

To achieve bright pupil images, the angular separation between camera and illuminator has to be very small. Such small separation is not only a huge challenge for lens and illuminator designs but also creates problematic internal reflection scenarios. In particular, under this camera/illuminator geometry, there are situations where the iris and pupil are imaged with approximately the same intensity, resulting in a very low pupil contrast. These situations are referred to as ‘grey pupil’ conditions. Any angular separation between above mentioned limits will create grey pupil conditions in some scenarios. Without a contrast between pupil and iris, there is no way to locate the pupil accurately.

Any discussion of the background art throughout the specification should in no way be considered as an admission that such art is widely known or forms part of common general knowledge in the field.

### SUMMARY OF THE INVENTION

In accordance with a first aspect of the present invention, there is provided a method for controlling one or more illumination devices in an eye tracker such that a measured pupil/iris contrast exceeds a predefined minimum pupil/iris contrast, the method including:
capturing images of a subject, including one or both of the subject's eyes, during predefined image capture periods;
illuminating, from one or more illumination devices, one or both of the subject's eyes during the predefined image capture periods, wherein at least one illumination device is located sufficiently close to a lens of the camera to generate bright pupil effects; and
selectively varying the output power of at least one of the illumination devices to generate a bright pupil reflection intensity such that a measured pupil/iris contrast in a captured image exceeds a predefined minimum pupil/iris contrast.

In some embodiments, the output power of at least one of the illumination devices is selectively varied based on a direct measure of pupil/iris contrast determined by pixel intensity of a pupil region relative to an iris region of one or both of the subject's eyes. In some embodiments, the output power of at least one of the illumination devices is selectively varied based on one or more of:
i. a measure of ambient light;
ii. a measure of pupil diameter of the subject; and/or
iii. a current or recent gaze direction of the subject.

In some embodiments, the measure of ambient light is obtained from an exposure setting of the camera and/or illumination settings of the one or more illumination devices.

In some embodiments, the output power of at least one of the illumination devices is selectively varied based on a measure of pupil contrast of the subject's eyes from a previous image capture period. In some embodiments, the output power of at least one of the illumination devices is selectively varied based on physiological parameters of the subject. In some embodiments, the output power of at least one of the illumination devices is selectively varied between at least two different power levels within an image capture period.

In some embodiments, the camera captures at least two images within an image capture period, and wherein the two images are captured with different illumination or image capture settings. In one embodiment, a first image is captured while the one or more illumination devices have a first power level and a second image is captured while the one or more illumination devices have a second power level different from the first power level.

In some embodiments the method includes the step of performing image subtraction on the two images to generate a resultant image of increased pupil contrast.

In some embodiments, the eye tracker includes a single illumination device. In some embodiments, the controller is configured to modulate the illumination power of the illumination device during an image capture period. In other embodiments, the eye tracker includes two illumination devices disposed at different distances from the camera. Preferably, each illumination device is located sufficiently close to a lens of the camera to generate bright pupil effects but at different distances from the lens to generate different bright pupil reflection characteristics.

In some embodiments, selectively varying the output power of at least one of the illumination devices includes deactivating one of the two illumination devices during an image capture period.

In accordance with a second aspect of the present invention, there is provided a method for controlling two or more illumination devices in an eye tracker such that a measured pupil/iris contrast exceeds a predefined minimum pupil/iris contrast, the method including:
capturing images of a subject, including one or both of the subject's eyes, during predefined image capture periods;
illuminating, from a system of two or more illumination devices, one or both of the subject's eyes during the predefined image capture periods, wherein each illumination device is located sufficiently close to a lens of the camera to generate bright pupil effects but at different distances from the lens to generate different bright pupil reflection characteristics; and
selectively varying the output power of the two or more illumination devices in order to generate a bright pupil reflection characteristic such that a measured pupil/iris contrast in a captured image exceeds a predefined minimum pupil/iris contrast.

In accordance with a third aspect of the present invention, there is provided a system for controlling one or more illumination devices in an eye tracker such that a measured pupil/iris contrast exceeds a predefined minimum pupil/iris contrast, the system including:
a camera configured to capture images of a subject, including one or both of the subject's eyes, during predefined image capture periods;
one or more illumination devices configured to selectively illuminate one or both of the subject's eyes during the predefined image capture periods, wherein at least one illumination device is located sufficiently close to a lens of the camera to generate bright pupil effects; and
a controller configured to selectively vary the output power of at least one of the illumination devices to generate a bright pupil reflection intensity such that a measured pupil/iris contrast in a captured image exceeds a predefined minimum pupil/iris contrast.

In some embodiments, the system includes one illumination device. Preferably the illumination device is located within a distance of 7 mm to 15 mm from the camera. More preferably, the illumination device is located within a distance of 8 mm to 14 mm from the camera. In other embodiments, the system includes two illumination devices. Preferably, each illumination device is located sufficiently close to a lens of the camera to generate bright pupil effects but at different distances from the lens to generate different bright pupil reflection characteristics.

In accordance with a fourth aspect of the present invention, there is provided a system for controlling two or more illumination devices in an eye tracker such that a measured pupil/iris contrast exceeds a predefined minimum pupil/iris contrast, the system including:
a camera configured to capture images of a subject, including one or both of the subject's eyes, during predefined image capture periods;
two or more illumination devices configured to selectively illuminate one or both of the subject's eyes during the predefined image capture periods, wherein each illumination device is located sufficiently close to a lens of the camera to generate bright pupil effects but at different distances from the lens to generate different bright pupil reflection characteristics; and
a controller configured to selectively vary the output power of the two or more illumination devices in order to generate a bright pupil reflection characteristic such that a measured pupil/iris contrast in a captured image exceeds a predefined minimum pupil/iris contrast.

In some embodiments, the system includes two illumination devices. Preferably, a first illumination device is disposed a distance of 3 mm to 15 mm from the camera while a second illumination device is disposed a distance of 7 mm to 50 mm from the camera. More preferably, the first illumination device is disposed a distance of 8 mm to 13 mm from the camera while a second illumination device is disposed a distance of 20 mm to 30 mm from the camera.

In some embodiments, the controller is configured to deactivate one of the illumination devices during an image capture period.

In some embodiments, the bright pupil reflection characteristics include a measure of a retroreflection effect by one or both of the subject's eyes. In some embodiments, the bright pupil reflection characteristics include a direct measure of pupil/iris contrast determined by pixel intensity of a pupil region relative to an iris region of one or both of the subject's eyes.

In accordance with a fifth aspect of the present invention, there is provided an illumination system for an eye tracker, the system including:
a camera configured to capture images of a subject, including one or both of the subject's eyes, during predefined image capture periods;
one or more illumination devices configured to illuminate one or both of the subject's eyes during the predefined image capture periods, wherein at least one illumination device is located sufficiently close to a lens of the camera to generate bright pupil effects; and
a controller configured to:
   process the captured images to measure a pupil contrast of the subject's eyes in at least a subset of the images; and
   control an output power of the one or more illumination devices based on a control signal to generate a bright pupil reflection intensity such that a measured pupil/iris contrast in a captured image exceeds a predefined minimum pupil/iris contrast, wherein the control signal is derived based on a measure of pupil contrast from a previous image capture period.

In accordance with a sixth aspect of the present invention, there is provided an eye tracking system including:
a camera configured to capture images of a subject, including one or both of the subject's eyes, during predefined image capture periods;
one or more illumination devices configured to illuminate one or both of the subject's eyes during the predefined image capture periods, wherein at least one illumination device is located sufficiently close to a lens of the camera to generate bright pupil effects; and
a controller configured to:
   process the captured images to perform an eye gaze tracking routine to track the eyes of the subject, the eye gaze tracking routine including determining one or more control parameters; and
   control an output power of the one or more illumination devices based on the one or more control parameters to generate a bright pupil reflection intensity such that a measured pupil/iris contrast in a captured image exceeds a predefined minimum pupil/iris contrast.

In some embodiments, the control parameters include:
i. a measure of ambient light;
ii. a measure of pupil diameter of the subject; and/or
iii. a current or recent gaze direction of the subject.

In some embodiments, the control parameters include physiological parameters of the subject.

In some embodiments, the processor is configured to process the captured images to determine a measure of pupil contrast of the subject's eyes and wherein the measure of pupil contrast from a previous image capture period is used as a control parameter to control an illumination power of the one or more illumination devices. Thus, the measure of pupil contrast from a previous image capture period may be a control parameter or factor upon which the illumination power of at least one of the illumination devices is selectively varied in any described embodiment.

### BRIEF DESCRIPTION OF THE FIGURES

Example embodiments of the disclosure will now be described, by way of example only, with reference to the accompanying drawings in which:

FIG. 1 is a perspective view of the interior of a vehicle having a driver monitoring system including a camera and two LED light sources installed therein;

FIG. 2 is a driver's perspective view of an automobile dashboard having the driver monitoring system of FIG. 1 installed therein;

FIG. 3 is a schematic functional view of a driver monitoring system according to FIGS. 1 and 2;

FIG. 4 is a schematic illustration of an eye being illuminated by an infrared illumination device to illustrate the bright pupil effect;

FIG. 5 is a schematic illustration of exemplary bright and dark pupil effect scenarios and associated images;

FIG. 6 is a plan view of the driver monitoring system of FIGS. 1 to 3 showing a camera field of view and an LED illumination field on a subject;

FIG. 7 is a plan view of the driver monitoring system of FIGS. 1 to 3 showing illumination and imaging geometry relative to a subject being imaged;

FIG. 8 is a process flow diagram illustrating the primary steps in an illumination method for an eye tracker;

FIG. 9 is an exemplary setup of a first embodiment of a camera and pair of LEDs;

FIG. 10 is a driver's perspective view of an automobile dashboard having a driver monitoring system with a single LED installed therein;

FIG. 11 is a simplified schematic functional view of the driver monitoring system of FIGS. 1 to 3 showing data flows between the different system components;

FIG. 12 illustrates exemplary scenarios where illumination from a far LED is preferable;

FIG. 13 illustrates exemplary scenarios where illumination from a near LED is preferable; and

FIG. 14 illustrates a graph of detected contrast between an iris and a pupil as a function of visual angle between IR light and a camera for four different pupil sizes.

### DESCRIPTION OF THE INVENTION

The illumination systems and methods described herein may be applied and used in a multitude of eye tracking environments. One example is monitoring a driver or passengers of an automobile or other vehicles such as a bus, train or airplane. Additionally, the described system may be applied to an operator using or operating any other equipment, such as machinery and flight simulators. For ease of understanding, the embodiments of the invention are described herein within the context of a driver monitoring system for a vehicle. Furthermore, although the illumination devices are described as being LEDs, it will be appreciated that the invention is applicable to other types of light sources such as vertical-cavity surface-emitting lasers (VCSELs).

### System Overview

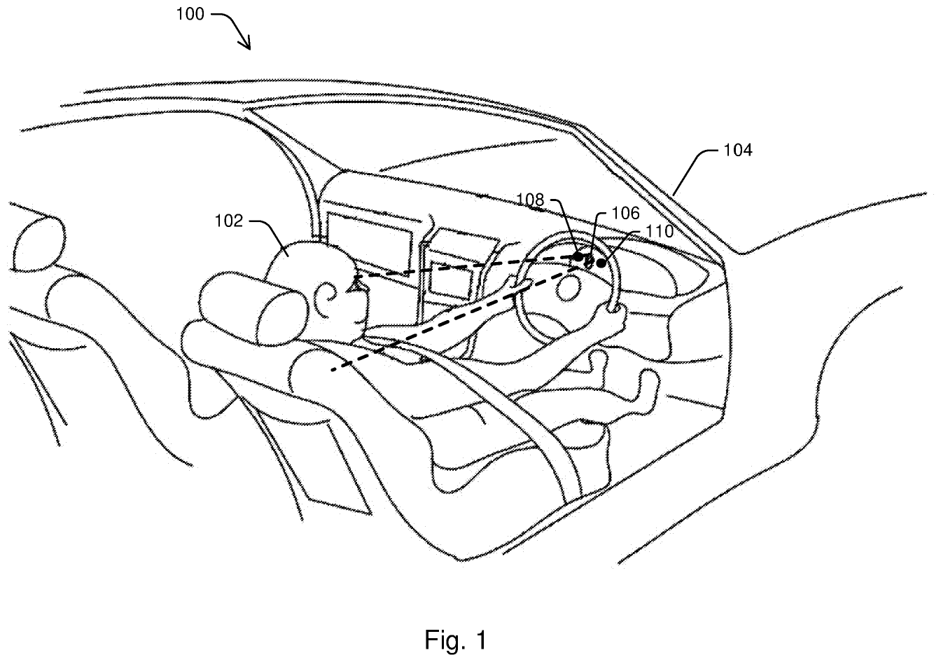

Referring initially to FIGS. 1 to 3, there is illustrated a driver monitoring system 100 for capturing images of a vehicle driver 102 during operation of a vehicle 104. System 100 is further adapted for performing various image processing algorithms on the captured images such as facial detection, facial feature detection, facial recognition, facial feature recognition, facial tracking or facial feature tracking, such as tracking a person's eyes. Example image processing routines are described in U.S. Pat. No. 7,043,056 to Edwards et al. entitled “Facial Image Processing System” and assigned to Seeing Machines Pty Ltd (hereinafter “Edwards et al.”), the contents of which are incorporated herein by way of cross-reference.

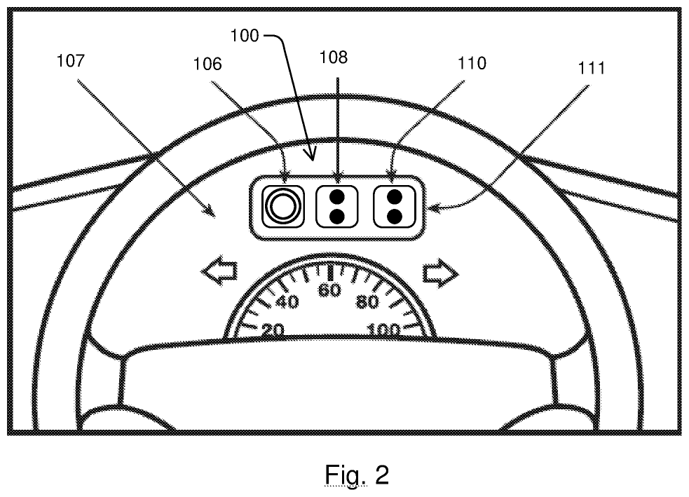

As best illustrated in FIG. 2, system 100 includes an imaging camera 106 that is positioned on or in the vehicle dash 107 instrument display and oriented to capture images of the driver's face in the infrared wavelength range to identify, locate and track one or more human facial features.

Camera 106 may be a conventional CCD or CMOS based digital camera having a two dimensional array of photosensitive pixels (or photosensor) and optionally the capability to determine range or depth (such as through one or more phase detect elements). The photosensitive pixels are capable of sensing electromagnetic radiation at least in the infrared range. Camera 106 may also be a three dimensional camera such as a time-of-flight camera or other scanning or range-based camera capable of imaging a scene in three dimensions. In other embodiments, camera 106 may be replaced by a pair of like cameras operating in a stereo configuration and calibrated to extract depth information of objects in images captured by camera 106. Although camera 106 is preferably configured to image in the infrared wavelength range, it will be appreciated that, in alternative embodiments, camera 106 may image in the visible range. Although not illustrated, camera 106 also includes a system of imaging optics which includes a primary imaging lens for focusing light onto the array of photosensitive pixels.

Referring still to FIG. 2, system 100, in a first embodiment, also includes a pair of infrared illumination devices in the form of light emitting diodes (LEDs) 108 and 110, horizontally disposed at respective positions proximate to the camera on vehicle dash 107. As will be described below, LEDs 108 and 110 are disposed at different distances from camera 106. In other embodiments described below, only a single LED is used. Also, in some embodiments, more than two light sources may be employed in the system.

LEDs 108 and 110 are adapted to illuminate driver 102 with infrared radiation, during predefined image capture periods when camera 106 is capturing an image, so as to enhance the driver's face to obtain high quality images of the driver's face or facial features. Operation of camera 106 and LEDs 108 and 110 in the infrared range reduces visual distraction to the driver. Operation of camera 106 and LEDs 108, 110 is controlled by an associated controller 112 which comprises a computer processor or microprocessor and memory for storing and buffering the captured images from camera 201. In other embodiments, different types of light sources such as VCSELs may be used in place of LEDs.

As best illustrated in FIG. 2, camera 106 and LEDs 108 and 110 may be manufactured or built as a single unit 111 having a common housing. The unit 111 is shown installed in a vehicle dash 107 and may be fitted during manufacture of the vehicle or installed subsequently as an after-market product. In other embodiments, the driver monitoring system 100 may include one or more cameras and light sources mounted in any location suitable to capture images of the head or facial features of a driver, subject and/or passenger in a vehicle. By way of example, cameras and LEDs may be located on a steering column, rearview mirror, center console or driver's side A-pillar of the vehicle. In the illustrated embodiment, the first and a second light source each include a single LED. In other embodiments, each light source may each include a plurality of individual LEDs.

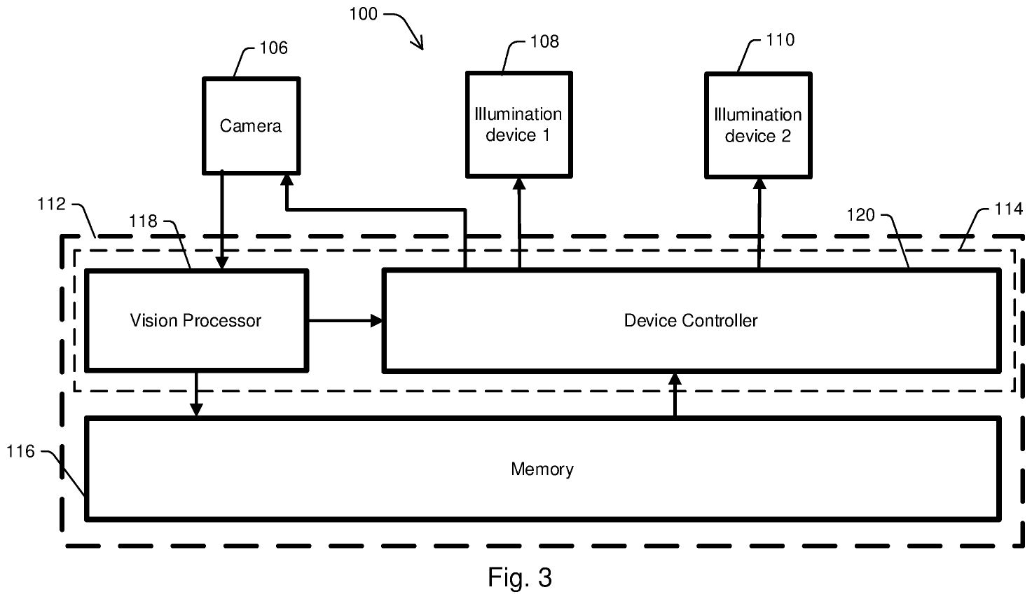

Turning now to FIG. 3, the functional components of system 100 are illustrated schematically. A system controller 112 acts as the central processor for system 100 and is configured to perform a number of functions as described below. Controller 112 is located within the dash 107 of vehicle 104 and may be connected to or integral with the vehicle on-board computer. In another embodiment, controller 112 may be located within a housing or module together with camera 106 and LEDs 108 and 110. The housing or module is able to be sold as an after-market product, mounted to a vehicle dash and subsequently calibrated for use in that vehicle. In further embodiments, such as flight simulators, controller 112 may be an external computer or unit such as a personal computer.

Controller 112 may be implemented as any form of computer processing device or portion of a device that processes electronic data, e.g., from registers and/or memory to transform that electronic data into other electronic data that, e.g., may be stored in registers and/or memory. As illustrated in FIG. 3, controller 112 includes a microprocessor 114, executing code stored in memory 116, such as random access memory (RAM), read-only memory (ROM), electrically erasable programmable read-only memory (EEPROM), and other equivalent memory or storage systems as should be readily apparent to those skilled in the art.

Microprocessor 114 of controller 112 includes a vision processor 118 and a device controller 120. Vision processor 118 and device controller 120 represent functional elements which are both performed by microprocessor 114. However, it will be appreciated that, in alternative embodiments, vision processor 118 and device controller 120 may be realized as separate hardware such as microprocessors in conjunction with custom or specialized circuitry.

Vision processor 118 is configured to process the captured images to perform the driver monitoring; for example, to determine a three dimensional head pose and/or eye gaze position of the driver 5 within the monitoring environment. To achieve this, vision processor 118 utilizes one or more eye gaze determination algorithms. This may include, by way of example, the methodology described in Edwards et al. Vision processor 118 may also perform various other functions including determining attributes of the driver 5 such as eye closure, blink rate and tracking the driver's head motion to detect driver attention, sleepiness or other issues that may interfere with the driver safely operating the vehicle.

The raw image data, gaze position data and other data obtained by vision processor 118 is stored in memory 116.

Device controller 120 is configured to control various parameters of camera 106 such as a shutter speed and/or image sensor exposure/integration time, and to selectively actuate LEDs 108 and 110 in a manner described below in sync with the exposure time of camera 106 or more generally within predefined image capture periods. LEDs 108 and 110 are preferably electrically connected to device controller 120 but may also be controlled wirelessly by controller 120 through wireless communication such as Bluetooth™ or WiFi™ communication.

Thus, during operation of vehicle 104, device controller 120 activates camera 106 to capture images of the face of driver 102 in a video sequence. LEDs 108 and 110 are activated and deactivated in synchronization with the image frames captured by camera 106 to illuminate the driver during predefined image capture periods. Working in conjunction, device controller 120 and vision processor 118 provide for capturing and processing images of the driver to obtain driver state information such as drowsiness, attention and gaze position during an ordinary operation of vehicle 104. Device controller 120 and vision processor 118 also work in conjunction to perform the dynamic illumination control described below.

Additional components of the system may also be included within the common housing of unit 111 or may be provided as separate components according to other additional embodiments. In one embodiment, the operation of controller 112 is performed by an onboard vehicle computer system which is connected to camera 106 and LEDs 108 and 112.

Throughout this specification, specific functions performed by vision processor 118 or device controller 120 may be described more broadly as being performed by controller 112.

### Bright Pupil Conditions

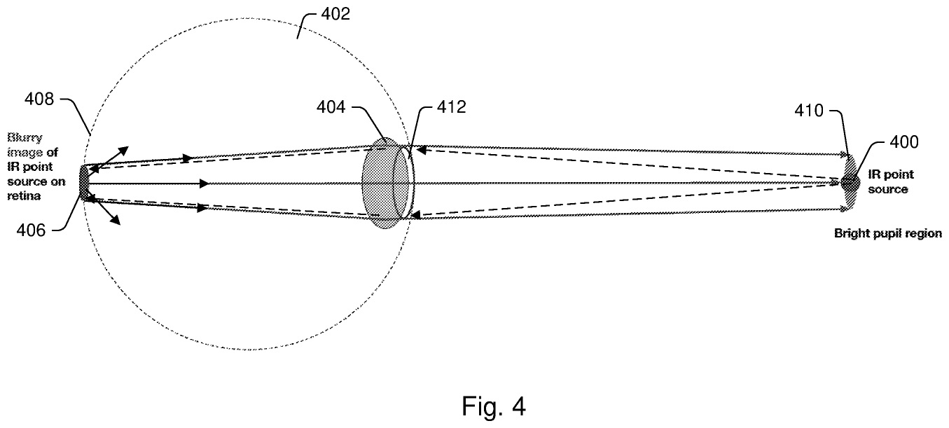

Referring now to FIG. 4, there is illustrated a schematic view of a bright pupil scenario. With reference to this figure, the concept of bright pupil conditions will be described.

When a point source such as an infrared LED 400 is used to illuminate an eye 402, the lens 404 of the eye 402 focusses the point source to an image 406 on the surface of the retina 408. As lens 404 is an imperfect lens, image 406 is a blurry disk on retina 408. Some light is diffusely reflected off retina 408 and a subset of this light is incident on lens 404. This subset of reflected light is focused by lens 404 back to the source. Again, as lens 404 is imperfect, the reflected light is focused to a disc of finite diameter around LED 400 rather than an ideal point. The disk represents a region 410 in which images captured will exhibit a bright pupil effect.

As only light that passes through the eye's pupil 412 is allowed into eye 402, the bright pupil region defined by region 410 becomes smaller with reduced brightness as the pupil diameter decreases. As the eye is rotated to view different regions in space (different gaze angles), the position, shape and size of image 406 on retina 408 changes. As a result, the size and intensity of region 410 changes with gaze angle.

The bright pupil effect is influenced by many factors including:
the dilation (size) of the pupil;
the gaze angle relative to the camera;
the age and ethnicity of the subject; and
the wavelength of light.

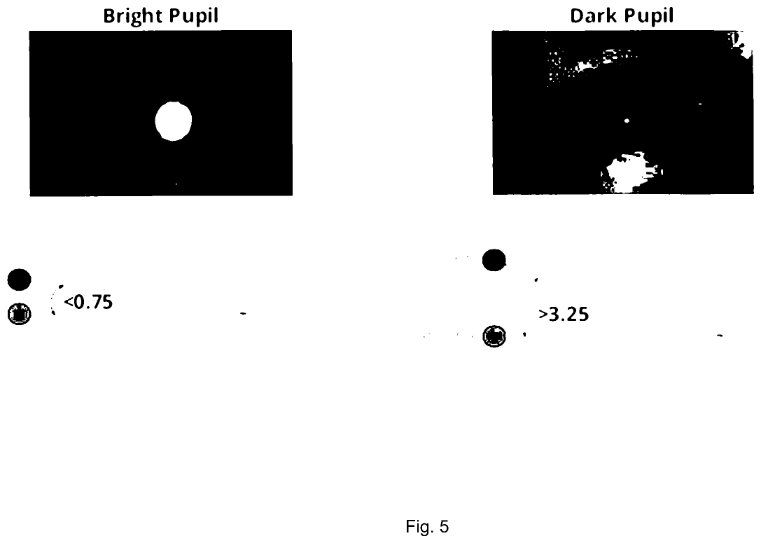

Under bright pupil effects, a pupil becomes brighter than the surrounding iris in a greyscale image. When imaged with a camera, the iris is seen as being a dark region around a significantly bright pupil. This is illustrated in the left panel of FIG. 5, which also shows an example angle at which the eye was illuminated. The right panel of FIG. 5 illustrates a more conventional dark pupil condition in which the pupil is darker than the surrounding iris.

Given the physiology of a human eye, the bright pupil effect is typically experienced when an imaging camera is located at an angle less than about 3.25 degrees from a light source with respect to the eye. However, it will be appreciated that the particular angles under which bright pupil effects are experienced vary with the above and other factors.

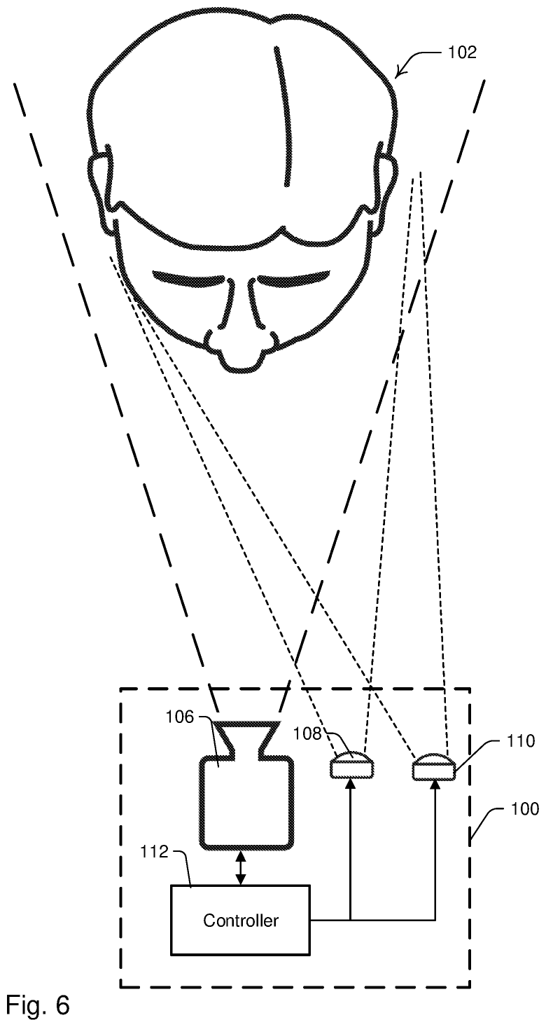

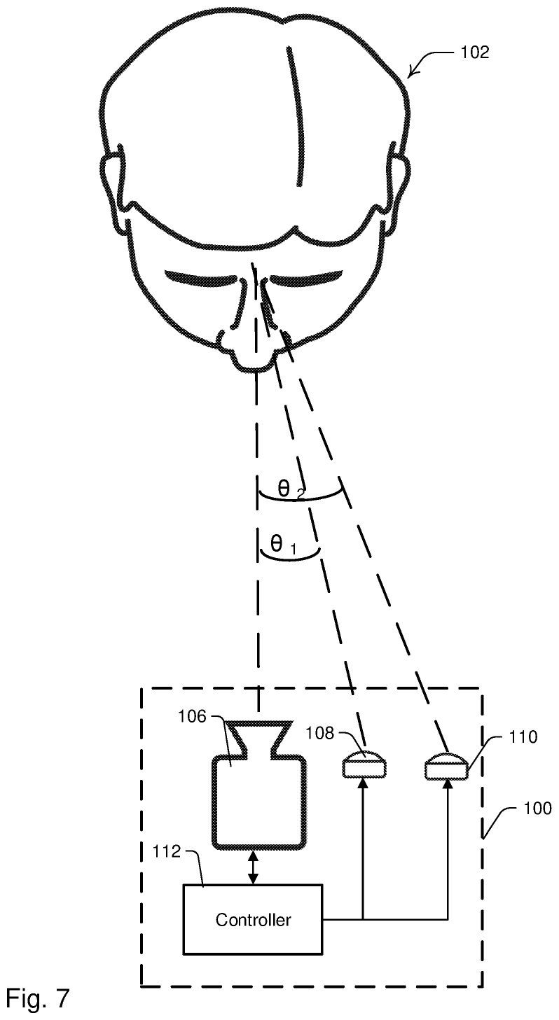

Referring now to FIGS. 6 and 7, there is illustrated a plan view of driver monitoring system 100. FIG. 6 illustrates the illumination fields of LEDs 108 and 110, and the field of view of camera 106. FIG. 7 illustrates the angular geometry between camera 106 and LEDs 108 and 110, including the camera optical axis. Under normal driving conditions, camera 106 and LEDs 108 and 110 are typically located a distance of between 30 cm and 80 cm from the face of driver 102. To achieve bright pupil conditions (with LEDs 108 and 110 illuminating driver 102 at angles less than 3.25 degrees (θ₁ and 0₂ in FIG. 7) from the optical axis of camera 106), LEDs 108 and 110 should be located within about 30 mm from camera 106. However, outer LED 110 may be located up to about 50 mm from the lens of camera 106.

For a given angular separation between a camera and illumination device, a larger pupil size will provide an increased pupil brightness. Similarly, for a given camera/illuminator angle, a subject staring directly at the camera will have darker pupils than when they look at off-axis directions.

When there is significant ambient illumination, the contrast between a bright pupil and the iris is determined by the brightness of the pupil and the brightness of the iris. The pupil brightness is due almost solely to the bright pupil effect from the controlled illumination from an LED. Ambient light does not add significantly to the intensity of the bright pupil. The brightness of the iris is due to a combination of the controlled illumination plus the ambient illumination.

Accordingly, it will be appreciated that, in other monitoring environments and under different conditions, LEDs 108 and 110 may be located at larger distances from camera 106 to still achieve bright pupil conditions.

As mentioned above, there are situations called ‘grey pupil’ conditions where the iris and pupil are imaged with approximately the same intensity, resulting in a very low pupil contrast. Typically, grey pupils only occur when the pupil size is small (when the pupil brightness is relatively low), thus they typically only occur in bright visible ambient conditions (when iris brightness is generally higher).

The grey pupil condition can be mitigated by:
Changing the intensity of the bright pupil effect (e.g. by moving the illumination source closer or further away from a lens of the imaging camera).
Adjusting the intensity of the controlled illumination.
Adjusting the intensity of the ambient light.

It is also possible to vary the pupil/iris contrast by varying an exposure period of the imaging camera, such as by changing the sensor integration time (shutter period). However, the inventors have identified that dynamically controlling the illumination power is advantageous as there is no added complexity or changes needed in the exposure control loop of the control algorithm of device controller 120. Dynamically controlling the illumination power is also advantageous to improve the signal to noise ratio in object tracking under poor light conditions as described in PCT Patent Application Publication WO 2019/084595 to Noble, entitled “System and Method for Improving Signal to Noise Ratio in Object Tracking Under Poor Light Conditions” and assigned to Seeing Machines Limited (hereinafter “Noble”). Dynamic illumination control may also be performed in conjunction with dynamic exposure control to improve system performance under some conditions but at the cost of additional complexity in the control algorithms.

### Illumination Control

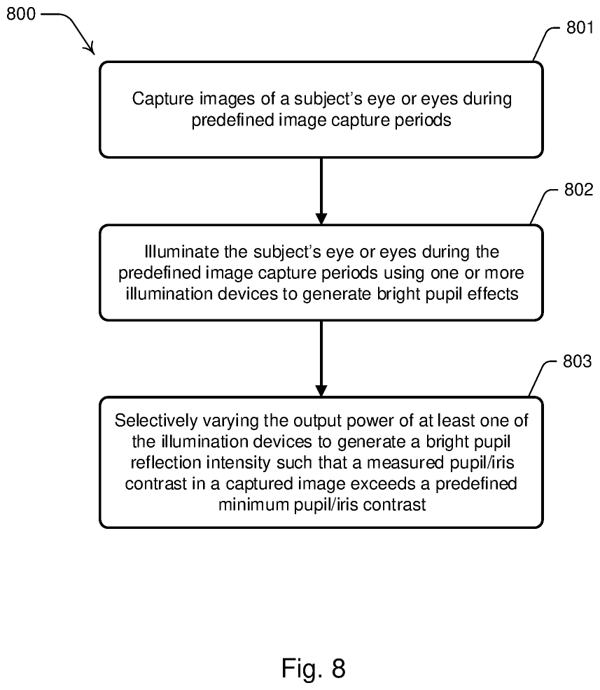

Referring now to FIG. 8, there is illustrated a process flow diagram showing the primary steps in an illumination method 800 for ensuring a minimum pupil/iris contrast between a pupil and an iris of an eye is maintained in an eye tracker system such as system 100. Method 800 includes, at step 801, configuring camera 106 to capture images of subject 102 during predefined image capture periods. The predefined image capture periods may represent a normal shutter period of camera 106 operating at a normal frame rate. However, camera 106 may be configured to operate in higher frame rate modes in which multiple integrations of an image sensor occur during a single shutter period. Similarly, some cameras are adapted to record multiple images within a single shutter period using multiple image sensors simultaneously storing image data. Here, the predefined image capture period may represent multiple periods of sensor integration. Thus, it is possible that multiple images are captured within a single image capture period. The terms “image capture periods” are intended to cover all variations of image capture, whether that be standard or higher frame rate sensor integration modes of camera 106.

The captured images include one or both of the subject's eyes. In some eye tracker systems, eye tracking of each eye is performed independently while, in other eye tracker systems, both eyes are tracked simultaneously. Where both eyes are not able to be tracked in certain image frames, a single eye may be tracked. Where neither eyes are able to be tracked (e.g. when the subject's eyes are closed), then that image frame may be discarded for the purpose of eye tracking and a subsequent image loaded.

At step 802, one or both of the subject's eyes are illuminated by one or both of LEDs 108 and 110 during the predefined image capture periods. In practice, steps 801 and 802 are performed synchronously such that the illumination occurs during the predefined image capture periods.

At step 803, controller 112 selectively varies the output power of at least one of LEDs 108 and 110 to generate a bright pupil reflection intensity such that a measured pupil/iris contrast in a captured image exceeds a predefined minimum pupil/iris contrast. As part of this step, vision processor 118 may calculate a pupil/iris contrast from a current or past images. This contrast calculation is described in detail below.

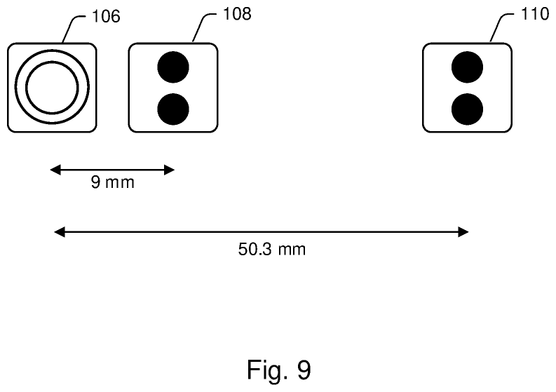

As described in more detail below, the present invention includes different embodiments having different combinations of LEDs (or other illumination devices). In a first embodiment, illustrated in FIGS. 1-3, 6 and 7, two LEDs (108 and 110) are provided and each LED is located sufficiently close to a lens of camera 106 so as to generate bright pupil effects but at different distances from the lens to generate different bright pupil reflection characteristics. By way of example, a setup of this first embodiment is illustrated schematically in FIG. 9, wherein LED 108 is located a distance of 9 mm from a lens of camera 106 and LED 110 is located a distance of 50.3 mm from the lens of camera 106.

It will be appreciated that other arrangements of LEDs 108 and 110 are possible to generate bright pupil effects and generate different bright pupil reflection characteristics. By way of example, near LED 108 may be positioned within a range of about 3 mm to 15 mm from the lens of camera 106 and far LED 110 may be positioned within a range of about 7 mm to 50 mm from the lens of camera 106. In some embodiments, it is preferable for near LED 108 to be positioned within a range of 8 mm to 13 mm and far LED 110 to be positioned within a range of 20 mm to 30 mm from the lens of camera 106.

It will also be appreciated that the optimal separations between camera 106 and LEDs 108 and 112 will depend on factors such as physical limitations of the camera lens and LED/secondary optics, and the presence and design of cover glass (to avoid excessive internal reflection).

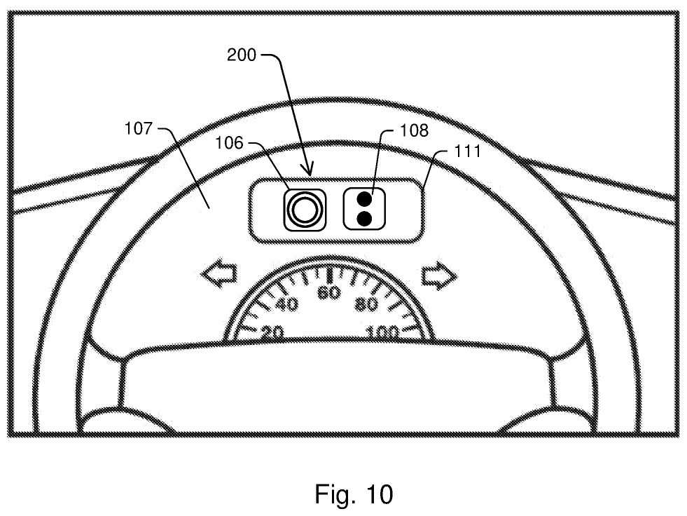

In a second embodiment system 200, illustrated in FIG. 10, only a single LED 108 is used to illuminate the subject's eye or eyes. The single LED 108 is located sufficiently close to a lens of camera 106 to generate bright pupil effects. In both embodiments, the LEDs are dynamically driven by controller 112 to selectively vary their output power to generate a bright pupil reflection intensity such that a measured pupil/iris contrast in a captured image exceeds a predefined minimum pupil/iris contrast. Achieving a minimum pupil/iris contrast increases the performance of an eye tracking system in being able to successfully detect and track the eye gaze of the subject being imaged.

Both of these embodiments are described below. It will be appreciated that each embodiment only relates to controlling LEDs (or illumination devices more generally) that are located sufficiently close to a lens of a camera so as to induce bright pupil effects. This is distinct from other illumination systems operating in dark pupil conditions where light sources may be located far from the lens of a camera and therefore significantly off the optical axis of the camera.

### Embodiment 1—Two LED Illumination

Referring again to FIG. 9, each illumination device is located sufficiently close to a lens of camera 106 to generate bright pupil effects (as described above) but at different distances from the lens to generate different bright pupil reflection characteristics. Bright pupil characteristics include a measure of the magnitude of the retroreflection effect experienced by the eye being illuminated. The magnitude of retroreflection effect is quantifiable by measuring pixel values of the iris and pupil at different illumination locations for a given LED power. This is measurable, in some embodiments, as a brightness or pixel intensity measure of one or more pixels within an iris region and one or more pixels within a pupil region within the captured images. These brightness measures are performed by vision processor 118 during the image processing of raw image data from camera 106.

The brightness measure is expressed in a greyscale brightness measure, typically in the format of the captured images (e.g. 16-bit). The brightness measure may include an average brightness value of a number of pixels determined to be located in each of the iris and pupil of an image. For example, in a dark pupil scenario, a raw 16-bit pupil brightness might be 3,540 while an iris brightness might be 18,050. In a bright pupil scenario, a raw 16-bit pupil brightness might be 32,501 while an iris brightness might be 3,460. The determination of the different regions of an eye is performed by vision processor 118 by known image processing techniques such as edge detection, shape recognition and contour detection.

Determination of an iris region and a pupil region may be performed as part of a broader eye tracking algorithm such as that described in Edwards et al. However, typically these regions may be identified by image processing techniques such as edge detection, shape recognition and Hough transforms. Thus, within an image, iris and pupil regions may be defined as a two dimensional region or regions of individual pixels.

A measure of pupil/iris contrast may be performed by comparing the brightness values of one or more pixels within a defined pupil region with brightness values of one or more pixels within a defined iris region. The sample of pixels may be a single pixel from each region, a group of pixels distributed around each region or an average pixel value of some or all of the pixels within each region.

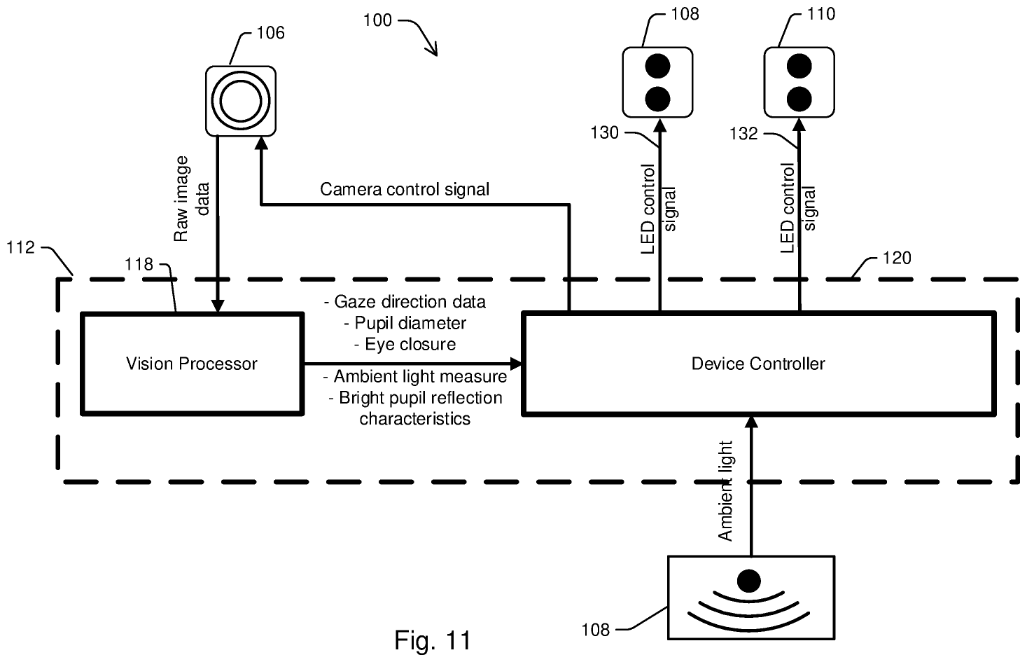

Referring now to FIG. 11, there is illustrated a variant of FIG. 3 showing the various data communicated between the different elements of system 100. Here, it is shown that vision processor 118 receives the raw image data from camera 106 and outputs various data to device controller 120. For simplicity, memory 116 is not illustrated in FIG. 11 but the data communication between camera 106, vision processor 118 and device controller 120 may involve storage and retrieval of data in memory 116.

During the illumination control step 803 of method 800, controller 112 controls an output power of one or both of LEDs 108 and 110 based on respective generated control signals 130 and 132. The control signals 130 and 132 are determined by device controller 120 and include a current and/or voltage signal at which the corresponding LED is driven to produce infrared radiation. Typically, LEDs are controlled with a pulsed output having a defined peak power and pulse duration. In system 100, the pulse duration is set by device controller 120 to substantially match the exposure time of camera 106.

The variation of output power by device controller 120 covers variations such as varying a pulse peak value, pulse shape or pulse duration. In some embodiments, the LEDs are controlled such that the energy content of the pulses is varied by device controller 120. In other embodiments, the energy content of the pulses may remain constant while a peak pulse value and/or pulse shape is varied. By way of example, the LEDs may be controlled dynamically based on a pulse handling curve as described in the Noble Publication referenced above.

The control signals determined by device controller 120 may be based on one or more of the following inputs:
A measure of ambient light. A measure of ambient light may be obtained from an external ambient light sensor 150 and fed to device controller 120. Alternatively, a measure of ambient light may be estimated by vision processor 118 from the captured images through comparison of background features over many images. Vision processor 118 may implement algorithms which extract a measure of ambient light from captured images by factoring in object reflectivity, distance to the object and an amount of controlled illumination. If the object being measured is fixed relative to the camera (such as a vehicle cabin object), then the distance factor remains constant. In some embodiments, a proxy measure of ambient light is obtained from exposure settings of camera 106 and/or illumination settings of the LEDs. As cameras have inbuilt hardware and software configured to control an image exposure time based on light conditions, using these exposure settings may be used as a proxy measure of ambient light. By way of example, in a scene with low ambient light conditions, camera 106 will automatically detect the light level via the image sensor and set a longer image exposure time.
A measure of pupil diameter of the subject. This is measured by vision processor 118 from a previous image where the pupil diameter is discernible. A larger diameter pupil indicates a lower level of ambient light while a smaller pupil indicates a higher level of ambient light. A measure of pupil diameter may also be used as a proxy measure of ambient light present.
A current or recent gaze direction of the subject. The complex geometry of a human eye means that different reflection characteristics are exhibited when light is directed onto different regions. When an eye is facing directly toward a light source, a high degree of retroreflection occurs and the bright pupil effect is exhibited. However, at different gaze angles away from a light source, different reflection characteristics are experienced which may or may not result in bright pupil effects. Moreover, these characteristics vary for each person as the eye geometry is different for humans. As such, a current or recent gaze direction measured by driver monitoring system 100 can be used as input to control LEDs 108 and 110.
Physiological parameters of the subject. Humans each have unique eye geometry with a variation in eye size among people of different age and ethnicity. In particular, the size of a human eye lens and amount of pupil dilation may vary among people. This gives rise to different bright and dark pupil responses for a given optical system. Example physiological parameters that vary among humans include the size, shape and reflectivity of the fundus or lens, and the shape and response of a pupil. In some embodiments, these parameters may be input to device controller 120 and considered in determining appropriate control signals 130 and 132. In some embodiments, these physiological parameters are directly measured. In other embodiments, the physiological parameters are fit by optimising the parameters based on training data or the like.
Direct measure of pupil contrast. In some embodiments, vision processor 118 is configured to process the captured images to determine a measure of pupil contrast of the subject's eyes. This measure of pupil contrast, which may be obtained from a current or past captured image, may form an input to device controller 120 for controlling the output power of LEDs 108 and 110. In some embodiments, the pupil contrast measure is obtained by comparing the brightness values of one or more pixels within a defined pupil region with brightness values of one or more pixels within a defined iris region, as described above. In other embodiments, the pupil contrast may be derived by other techniques such as determining a slope of pixel values across the iris and pupil regions.

Device controller 120 includes one or more algorithms which produce desired control signals 130 and 132 for LEDs 108 and 110 based on one or more of the above inputs. The specific voltage or current values of the control signals may be based on a combination of the measured inputs described above as determined by the control algorithms. In some embodiments, a controller 120 runs a specific control algorithm that sets a desired voltage and/or current for LEDs 108 and 110 based on the measured inputs. In some embodiments, each input is weighted based on importance.

Typically, in a dark environment (where the ambient light is low), near LED 108 can be activated while deactivating far LED 110. For dilated pupils, the upper limit of angular separation between camera and LED can be larger, which will mitigate the internal reflection problem. For scenarios where the pupil size is small but the subject is staring off-axis or where the pupil size is sufficiently large, it is preferable to activate the close LED 108 to enhance the pupil/iris contrast.

Typically, in bright environments (where the ambient light is high), far LED 110 can be activated while near LED 108 is deactivated. For constricted pupils, the lower limit of angular separation between camera 106 and LEDs 108 and 110 can be smaller, which will be translated to more compact size of camera assembly. Far LED 110 can also be activated for scenarios when the subject is staring at camera 106 or where the pupil size is sufficiently small.

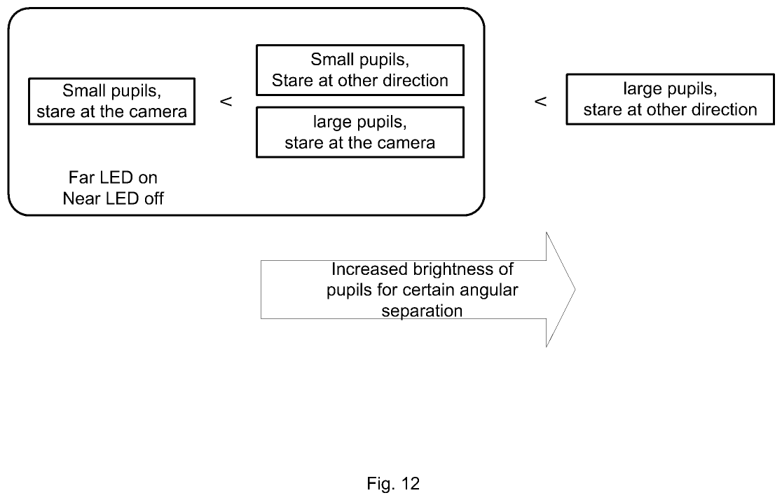

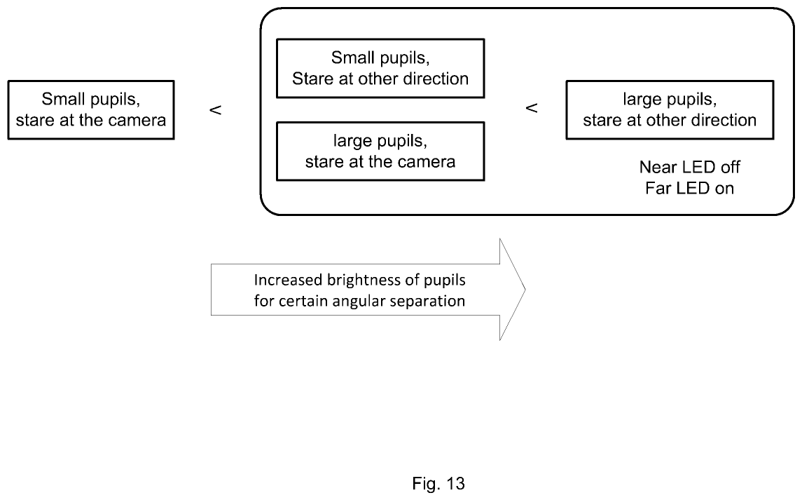

FIG. 12 illustrates exemplary scenarios where illumination from far LED 110 is preferable while FIG. 13 illustrates exemplary scenarios where illumination from near LED 108 is preferable. Note, there are some overlapping scenarios between FIGS. 12 and 13. This means for some scenarios, either the near LED 108 or far LED 110 can be activated. For given LED positions and pupil size, if a subject stares directly at the camera, their pupils will be darker than that when they look at other direction.

Thus, at step 803, device controller may implement control signals 130 and 132 such that the following exemplary illumination conditions are provided:
LED 108 is activated while LED 110 is deactivated.
LED 110 is activated while LED 108 is deactivated.
The output power of LED 108 is increased while LED 110 is deactivated.
The output power of LED 110 is increased while LED 108 is deactivated.
The output power of LED 108 is increased while LED 110 is decreased.
The output power of LED 110 is increased while LED 108 is decreased.
The output power of LED 108 is decreased while LED 110 is deactivated.
The output power of LED 110 is decreased while LED 108 is deactivated.

It will be appreciated that the above illumination conditions are exemplary only and are not an exhaustive list of possibilities. The amount that each LED is increased or decreased may be based on a combination of the above inputs. In some embodiments, each LED is driveable at one of a number of predefined voltage or current levels based on the specific values or ranges of the detected inputs. In some embodiments, device controller 120 varies the illumination power of the one or more illumination devices between at least two different power levels within an image capture period.

It will be appreciated that, in other embodiments, system 100 includes more than two LEDs positioned to generate bright pupil effects.

### Embodiment 2—Single LED

Referring now to FIG. 10, system 200 represents a second embodiment in which only near LED 108 is implemented. In this embodiment, LED 108 is preferably, but not necessarily, located at a distance of 3 mm to 15 mm from the lens of camera 106.

This single LED embodiment relies on the fact that the presence of a grey pupil effect can be mitigated without changing illumination angle by simply dynamically adjusting the controlled illumination intensity of an LED until the ratio between ambient and controlled light restores the pupil/iris contrast to a minimum level. Here, controlled light represents the amount of light that is generated in a controlled manner from LED 108 while ambient light represents all other light imaged by the photosensor array of camera 106.

The dynamic control of LED 108 (and also LED 110 in the case of the first embodiment) may be performed by device controller 120 based on the understanding that:
i. In low ambient light conditions, the pupil is large but decreases in size as the ambient light increases.
ii. The iris becomes brighter with increased ambient light while the pupil does not.
iii. The controlled illumination by LED 108 (and/or LED 110) will be reduced by an automatic camera exposure control algorithm executed by device controller 120, which will reduce the brightness of the pupil and iris by the same proportion. I.e. the brightness of the pupil will decrease by a larger absolute amount since it started at a higher value. This will reduce the absolute contrast between the pupil and iris.

The combination of the above three effects means that, under bright pupil conditions, the pupil size decreases which reduces the bright pupil effect (however there is still a usable bright pupil effect down to a small pupil diameter under very low ambient conditions). At the same time, increasing ambient and LED output power control algorithm adjustments combine to reduce the brightness of the pupil and increase the brightness of the iris. All these effects combined act to significantly reduce the bright pupil effect and cause the grey pupil effect to appear at smaller illumination angles.

Thus, the LED output power control algorithm should take into account these variations in pupil size and iris brightness with ambient conditions. Further, it is likely that there will be no LED location that can successfully create a bright pupil across all ambient conditions and resulting pupil size and controlled illumination levels.

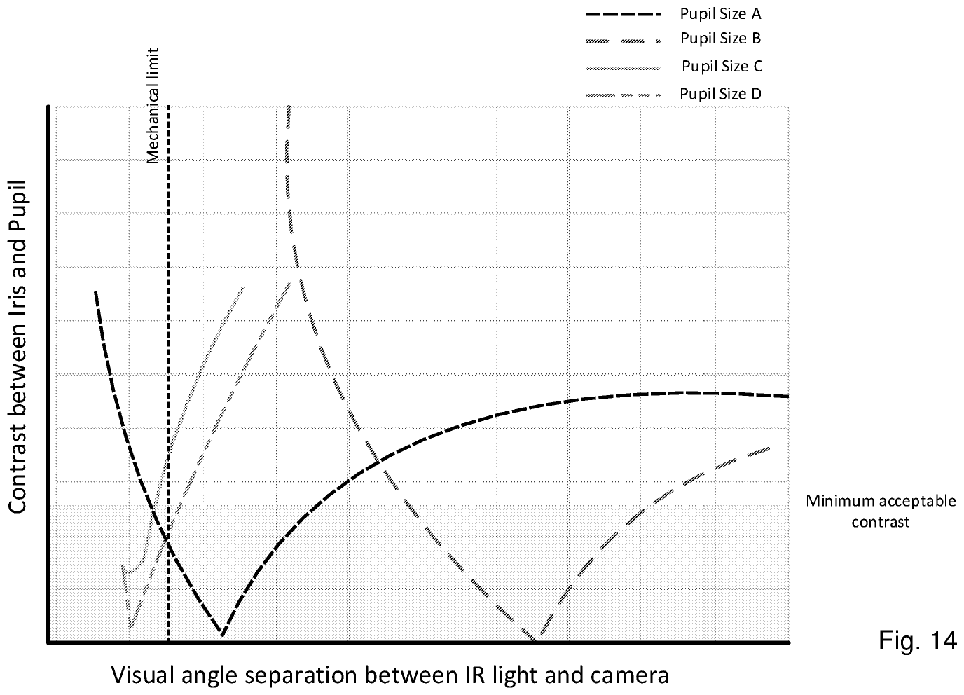

Referring now to FIG. 14, there is illustrated a graph of detected iris/pupil contrast as a function of angle between LED and camera lens for four different pupil sizes. This graph illustrates a relationship between pupil size, pupil/iris contrast and LED/camera separation for a given distance. These relationships allow for estimating a level of ambient light and for a set of rules to be developed for controlling the LED(s) to achieve a desired iris/pupil contrast.

In some embodiments, camera 106 is adapted to capture multiple images per image capture period. In these embodiments, device controller 120 may control LED 108 to capture at least two images within an image capture period under different illumination conditions and/or image capture conditions, say by modulating the output power of LED 108. For example, one image may be captured while LED 108 is driven at a first power level and a second image is captured while LED 108 is driven at a second output power level different from the first power level. This results in two simultaneous or very closely temporally spaced apart images having different levels of controlled light but a common level of ambient light. In other embodiments, image capture settings such as the exposure time or sensor gain can be modified between images.

Vision processor 118 may then perform image subtraction on the two images to generate a resultant image of increased pupil contrast. In this image subtraction process, pixel values of corresponding pixels of the two images are subtracted to remove the ambient light component and enhance the bright pupil effect.

A similar image subtraction process may be performed for the first embodiment with one or both LEDs 108 and 110 being modulated at different output power levels.

The above described invention can provide efficient eye tracking using pupil/iris contrast with equivalent performance to a standard eye tracking system operating in dark pupil mode but with a 50% decrease in package size (for the first embodiment). This reduced size is advantageous in modern vehicles where space on a dashboard instrument panel is a valuable commodity. In the case of the second embodiment (single LED), the package size can be further reduced with a performance reduction in circumstances where the pupil size is small.

### INTERPRETATION

The term “infrared” is used throughout the description and specification. Within the scope of this specification, infrared refers to the general infrared area of the electromagnetic spectrum which includes near infrared, infrared and far infrared frequencies or light waves.

Unless specifically stated otherwise, as apparent from the following discussions, it is appreciated that throughout the specification discussions utilizing terms such as “processing,” “computing,” “calculating,” “determining”, analyzing” or the like, refer to the action and/or processes of a computer or computing system, or similar electronic computing device, that manipulate and/or transform data represented as physical, such as electronic, quantities into other data similarly represented as physical quantities.

In a similar manner, the term “controller” or “processor” may refer to any device or portion of a device that processes electronic data, e.g., from registers and/or memory to transform that electronic data into other electronic data that, e.g., may be stored in registers and/or memory. A “computer” or a “computing machine” or a “computing platform” may include one or more processors.

Reference throughout this specification to “one embodiment”, “some embodiments” or “an embodiment” means that a particular feature, structure or characteristic described in connection with the embodiment is included in at least one embodiment of the present disclosure. Thus, appearances of the phrases “in one embodiment”, “in some embodiments” or “in an embodiment” in various places throughout this specification are not necessarily all referring to the same embodiment. Furthermore, the particular features, structures or characteristics may be combined in any suitable manner, as would be apparent to one of ordinary skill in the art from this disclosure, in one or more embodiments.

As used herein, unless otherwise specified the use of the ordinal adjectives “first”, “second”, “third”, etc., to describe a common object, merely indicate that different instances of like objects are being referred to, and are not intended to imply that the objects so described must be in a given sequence, either temporally, spatially, in ranking, or in any other manner.

In the claims below and the description herein, any one of the terms comprising, comprised of or which comprises is an open term that means including at least the elements/features that follow, but not excluding others. Thus, the term comprising, when used in the claims, should not be interpreted as being limitative to the means or elements or steps listed thereafter. For example, the scope of the expression a device comprising A and B should not be limited to devices consisting only of elements A and B. Any one of the terms including or which includes or that includes as used herein is also an open term that also means including at least the elements/features that follow the term, but not excluding others. Thus, including is synonymous with and means comprising.

It should be appreciated that in the above description of exemplary embodiments of the disclosure, various features of the disclosure are sometimes grouped together in a single embodiment, Fig., or description thereof for the purpose of streamlining the disclosure and aiding in the understanding of one or more of the various inventive aspects. This method of disclosure, however, is not to be interpreted as reflecting an intention that the claims require more features than are expressly recited in each claim. Rather, as the following claims reflect, inventive aspects lie in less than all features of a single foregoing disclosed embodiment. Thus, the claims following the Detailed Description are hereby expressly incorporated into this Detailed Description, with each claim standing on its own as a separate embodiment of this disclosure.

Furthermore, while some embodiments described herein include some but not other features included in other embodiments, combinations of features of different embodiments are meant to be within the scope of the disclosure, and form different embodiments, as would be understood by those skilled in the art. For example, in the following claims, any of the claimed embodiments can be used in any combination.

In the description provided herein, numerous specific details are set forth. However, it is understood that embodiments of the disclosure may be practiced without these specific details. In other instances, well-known methods, structures and techniques have not been shown in detail in order not to obscure an understanding of this description.

Similarly, it is to be noticed that the term coupled, when used in the claims, should not be interpreted as being limited to direct connections only. The terms “coupled” and “connected,” along with their derivatives, may be used. It should be understood that these terms are not intended as synonyms for each other. Thus, the scope of the expression a device A coupled to a device B should not be limited to devices or systems wherein an output of device A is directly connected to an input of device B. It means that there exists a path between an output of A and an input of B which may be a path including other devices or means. “Coupled” may mean that two or more elements are either in direct physical, electrical or optical contact, or that two or more elements are not in direct contact with each other but yet still co-operate or interact with each other.

Embodiments described herein are intended to cover any adaptations or variations of the present invention. Although the present invention has been described and explained in terms of particular exemplary embodiments, one skilled in the art will realize that additional embodiments can be readily envisioned that are within the scope of the present invention.

1. A method for controlling one or more illumination devices in an eye tracker such that a measured pupil/iris contrast exceeds a predefined minimum pupil/iris contrast, the method comprising:
capturing images of a subject, including one or both of the subject's eyes, during predefined image capture periods;
illuminating, from one or more illumination devices, one or both of the subject's eyes during the predefined image capture periods, wherein at least one illumination device is located sufficiently close to a lens of the camera to generate bright pupil effects; and
selectively varying the output power of at least one of the illumination devices to generate a bright pupil reflection intensity such that a measured pupil/iris contrast in a captured image exceeds a predefined minimum pupil/iris contrast.

2. The method according to claim 1 wherein the output power of at least one of the illumination devices is selectively varied based on a direct measure of pupil/iris contrast determined by pixel intensity of a pupil region relative to an iris region of one or both of the subject's eyes.

3. The method according to claim 1 wherein the output power of at least one of the illumination devices is selectively varied based on one or more of:
i. a measure of ambient light;
ii. a measure of pupil diameter of the subject; and/or
iii. a current or recent gaze direction of the subject.

4. The method according to claim 3 wherein the measure of ambient light is obtained from an exposure setting of the camera and/or illumination settings of the one or more illumination devices.

5. The method according to claim 1 wherein the output power of at least one of the illumination devices is selectively varied based on physiological parameters of the subject.

6. The method according to claim 1 wherein the output power of at least one of the illumination devices is selectively varied between at least two different power levels within an image capture period.

7. The method according to claim 6 wherein the camera captures at least two images within an image capture period, and wherein the two images are captured with different illumination or image capture settings.

8. The method according to claim 7 including the step of performing image subtraction on the two images to generate a resultant image of increased pupil contrast.

9. The method according to claim 1 wherein the eye tracker includes a single illumination device.

10. The method according to claim 1 wherein the controller is configured to modulate the illumination power of the one or more illumination devices during an image capture period.

11. The method according to claim 1 wherein the eye tracker includes two illumination devices disposed at different distances from the camera.

12. The method according to claim 11 wherein selectively varying the output power of at least one of the illumination devices includes deactivating one of the two illumination devices during an image capture period.

13. The method according to claim 11 wherein each illumination device is located sufficiently close to a lens of the camera to generate bright pupil effects but at different distances from the lens to generate different bright pupil reflection characteristics.

14. A system for controlling one or more illumination devices in an eye tracker such that a measured pupil/iris contrast exceeds a predefined minimum pupil/iris contrast, the system comprising:
a camera configured to capture images of a subject, including one or both of the subject's eyes, during predefined image capture periods;
one or more illumination devices configured to selectively illuminate one or both of the subject's eyes during the predefined image capture periods, wherein at least one illumination device is located sufficiently close to a lens of the camera to generate bright pupil effects; and
a controller configured to selectively vary the output power of at least one of the illumination devices to generate a bright pupil reflection intensity such that a measured pupil/iris contrast in a captured image exceeds a predefined minimum pupil/iris contrast.

15. The system according to claim 14 including one illumination device.

16. The system according to claim 14 including two illumination devices, wherein each illumination device is located sufficiently close to a lens of the camera to generate bright pupil effects but at different distances from the lens to generate different bright pupil reflection characteristics.

17. The system according to claim 16 wherein a first illumination device is disposed a distance of 3 mm to 15 mm from the camera while a second illumination device is disposed a distance of 7 mm to 50 mm from the camera.

18. The system according to claim 16 wherein the controller is configured to deactivate one of the illumination devices during an image capture period.

19. The system according to claim 14 wherein the bright pupil reflection characteristics include a direct measure of pupil/iris contrast determined by pixel intensity of a pupil region relative to an iris region of one or both of the subject's eyes.

20. An eye tracking system comprising:
a camera configured to capture images of a subject, including one or both of the subject's eyes, during predefined image capture periods;
one or more illumination devices configured to illuminate one or both of the subject's eyes during the predefined image capture periods, wherein at least one illumination device is located sufficiently close to a lens of the camera to generate bright pupil effects; and
a controller configured to:
process the captured images to perform an eye gaze tracking routine to track the eyes of the subject, the eye gaze tracking routine including determining one or more control parameters; and
control an output power of the one or more illumination devices based on the one or more control parameters to generate a bright pupil reflection intensity such that a measured pupil/iris contrast in a captured image exceeds a predefined minimum pupil/iris contrast.

21. (canceled)

22. The method according to claim 1 wherein the output power of at least one of the illumination devices is selectively varied based on a measure of pupil contrast of the subject's eyes from a previous image capture period.
